SEQUENCE LISTING (45 sequences)

SEQ ID NO: 1 - protein, 50 aa, Artificial Sequence
  VP64
DALDDFDLDMLGSDALDDFDLDMLGSDALDDFDLDMLGSDALDDFDLDML

SEQ ID NO: 2 - protein, 113 aa, Human herpesvirus 4
PAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 3 - protein, 86 aa, Human herpesvirus 4
REMADTVIPQKEEAAICGQMDLSHPPPRGHLDELTTTLESMTEDLNLDSPLTPELNEILDTFLNDECLLHAMHISTGLSIFDTSLF

SEQ ID NO: 4 - protein, 605 aa, Human herpesvirus 4
MRPKKDGLEDFLRLTPEIKKQLGSLVSDYCNVLNKEFTAGSVEITLRSYKICKAFINEAKAHGREWGGLMATLNICNFWAILRNNRVRRRAENAGNDACSIACPIVMRYVLDHLIVVTDRFFIQAPSNRVMIPATIGTAMYKLLKHSRVRAYTYSKVLGVDRAAIMASGKQVVEHLNRMEKEGLLSSKFKAFCKWVFTYPVLEEMFQTMVSSKTGHLTDDVKDVRALIKTLPRASYSSHAGQRSYVSGVLPACLLSTKSKAVETPILVSGADRMDEELMGNDGGASHTEDRYSESGQFHAFTDELESLPSPTMPLKPGAQSADCGDSSSSSSDSGNSDTEQSEREEARAEAPRLRAPKSRRTSRPNRGQTPCSSNAEEPEQPWIAAVHQESDERPIFPHPSKPTFLPPVKRKKGLRDSREGMFLPKPEAGSAISDVFEGREVCQPKRIRPFHPPGSPWANRPLPASLAPTPTGPVHEPIGSLTPASVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 5 - DNA, 501 bp, Artificial Sequence
  VP64-miniRTA
  CDS (1)..(501)
gatgctttagacgattttgacttagatatgcttggttcagacgcgttagacgacttcgacctagacatgttaggctcagatgcattggacgacttcgatttagatatgttgggctccgatgccctagatgactttgatttggatatgctaggatctggtagcccagcgcccgcagtgactcccgaggccagtcacctgttggaagatcccgatgaagagaccagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 6 - protein, 167 aa, Artificial Sequence
  Synthetic Construct
DALDDFDLDMLGSDALDDFDLDMLGSDALDDFDLDMLGSDALDDFDLDMLGSGSPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 7 - DNA, 420 bp, Artificial Sequence
  VP64-microRTA
  CDS (1)..(420)
gatgcactcgatgattttgacctcgatatgcttgggagtgatgcgctcgatgacttcgatttggatatgcttggatctgatgccctcgacgatttcgaccttgatatgctcgggtcagacgctttggatgactttgaccttgacatgctggggagcggctcccgggagatggctgacacagtaataccccaaaaagaggaggctgcgatttgtgggcagatggatttgtcccaccctccaccgagaggtcatcttgacgaattgacaacgacgctcgaatccatgaccgaggacctgaacctcgatagcccgctcacccccgagttgaatgagatcctggatacatttcttaatgatgagtgtttgcttcacgcaatgcatatttctacgggtcttagtattttcgacacgagcctgttt

SEQ ID NO: 8 - protein, 140 aa, Artificial Sequence
  Synthetic Construct
DALDDFDLDMLGSDALDDFDLDMLGSDALDDFDLDMLGSDALDDFDLDMLGSGSREMADTVIPQKEEAAICGQMDLSHPPPRGHLDELTTTLESMTEDLNLDSPLTPELNEILDTFLNDECLLHAMHISTGLSIFDTSLF

SEQ ID NO: 9 - DNA, 462 bp, Artificial Sequence
  VP64-MyoD
  CDS (1)..(462)
gatgctttagacgattttgacttagatatgcttggttcagacgcgttagacgacttcgacctagacatgttaggctcagatgcattggacgacttcgatttagatatgttgggctccgatgccctagatgactttgatttggatatgctaggatctggtagcatggagctactgtcgccaccgctccgcgacgtagacctgacggcccccgacggctctctctgctcctttgccacaacggacgacttctatgacgacccgtgtttcgactccccggacctgcgcttcttcgaggacctggacccgcgcctgatgcacgtgggcgcgctcctgaaacccgaagagcactcgcacttccctgcggctgttcacccggcaccgggggcacgcgaggacgaacatgtcagggctcccagcggtcatcaccaggctggtcggtgtctgttgtgggcctgcaaggcg

SEQ ID NO: 10 - protein, 154 aa, Artificial Sequence
  Synthetic Construct
DALDDFDLDMLGSDALDDFDLDMLGSDALDDFDLDMLGSDALDDFDLDMLGSGSMELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKA

SEQ ID NO: 11 - DNA, 462 bp, Artificial Sequence
  VP64-HSF1
  CDS (1)..(462)
gatgctttagacgattttgacttagatatgcttggttcagacgcgttagacgacttcgacctagacatgttaggctcagatgcattggacgacttcgatttagatatgttgggctccgatgccctagatgactttgatttggatatgctaggtagcagtgggcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 12 - protein, 154 aa, Artificial Sequence
  Synthetic Construct
DALDDFDLDMLGSDALDDFDLDMLGSDALDDFDLDMLGSDALDDFDLDMLGSSGPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 13 - DNA, 480 bp, Artificial Sequence
  VP32-p65
  CDS (1)..(480)
gatgcattggacgacttcgatttagatatgttgggctccgatgccctagatgactttgatttggatatgctaggatctggtagccctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 14 - protein, 160 aa, Artificial Sequence
  Synthetic Construct
DALDDFDLDMLGSDALDDFDLDMLGSGSPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 15 - DNA, 558 bp, Artificial Sequence
  VP64-p65
  CDS (1)..(558)
gatgctttagacgattttgacttagatatgcttggttcagacgcgttagacgacttcgacctagacatgttaggctcagatgcattggacgacttcgatttagatatgttgggctccgatgccctagatgactttgatttggatatgctaggatctggtagccctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 16 - protein, 186 aa, Artificial Sequence
  Synthetic Construct
DALDDFDLDMLGSDALDDFDLDMLGSDALDDFDLDMLGSDALDDFDLDMLGSGSPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 17 - DNA, 1128 bp, Artificial Sequence
  VPH
  CDS (1)..(1128)
gatgctttagacgattttgacttagatatgcttggttcagacgcgttagacgacttcgacctagacatgttaggctcagatgcattggacgacttcgatttagatatgttgggctccgatgccctagatgactttgatttggatatgctaagttccggatctccgaaaaagaaacgcaaagttggtagcccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagacctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 18 - protein, 376 aa, Artificial Sequence
  Synthetic Construct
DALDDFDLDMLGSDALDDFDLDMLGSDALDDFDLDMLGSDALDDFDLDMLSSGSPKKKRKVGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 19 - DNA, 1530 bp, Artificial Sequence
  VPR
  CDS (1)..(1530)
gacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 20 - protein, 510 aa, Artificial Sequence
  Synthetic Construct
DALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 21 - protein, 11 aa, human herpesvirus 1
DALDDFDLDML

SEQ ID NO: 22 - protein, 4 aa, Artificial Sequence
  peptide linker
GSGS

SEQ ID NO: 23 - protein, 4 aa, Artificial Sequence
  peptide linker
GSSG

SEQ ID NO: 24 - protein, 5 aa, Artificial Sequence
  peptide linker
GGGGS

SEQ ID NO: 25 - protein, 5 aa, Artificial Sequence
  peptide linker
GGGAR

SEQ ID NO: 26 - protein, 6 aa, Artificial Sequence
  peptide linker
GSGSGS

SEQ ID NO: 27 - protein, 9 aa, Artificial Sequence
  peptide linker
SGQGGGGSG

SEQ ID NO: 28 - DNA, 3162 bp, Staphylococcus aureus
  CDS (1)..(3162)
  gene (1)..(3162): dSaCas9
atgaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 29 - protein, 1053 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 30 - RNA, 83 nt, Staphylococcus aureus
  misc_structure (1)..(83): tracrRNA
guuuuaguacucuggaaacagaaucuacuaaaacaaggcaaaaugccguguuuaucacgucaacuuguuggcgagauuuuuuu

SEQ ID NO: 31 - RNA, 14 nt, Staphylococcus aureus
  misc_structure (1)..(14): repeat region of crRNA
guuuuaguacucug

SEQ ID NO: 32 - RNA, 16 nt, Staphylococcus aureus
  misc_structure (1)..(16): anti-repeat region of tracrRNA
cagaaucuacuaaaac

SEQ ID NO: 33 - RNA, 49 nt, Staphylococcus aureus
  misc_structure (1)..(49): stem loop 1 region, linker region and stem loop 2 region
aaggcaaaaugccguguuuaucacgucaacuuguuggcgagauuuuuuu

SEQ ID NO: 34 - RNA, 19 nt, Lachnospiraceae bacterium
  misc_structure (1)..(19): 5′ handle of crRNA
aauuucuacucuuguagau

SEQ ID NO: 35 - DNA, 21 bp, Homo sapiens
ggttcatacggtcctgccctc

SEQ ID NO: 36 - DNA, 21 bp, Homo sapiens
ggagccacagttcttccacgg

SEQ ID NO: 37 - DNA, 21 bp, Homo sapiens
ctctacccttgaggtctcgag

SEQ ID NO: 38 - DNA, 21 bp, Homo sapiens
tgccagattccagttgtccag

SEQ ID NO: 39 - DNA, 21 bp, Homo sapiens
acattcctgagtctcagagag

SEQ ID NO: 40 - DNA, 21 bp, Homo sapiens
ggctaatttcctggagcccct

SEQ ID NO: 41 - DNA, 21 bp, Homo sapiens
ctgtgaggctaaacagagctg

SEQ ID NO: 42 - DNA, 21 bp, Homo sapiens
gtctctcacccaatataagca

SEQ ID NO: 43 - DNA, 21 bp, Homo sapiens
aaatcacttaagttctctaaa

SEQ ID NO: 44 - protein, 128 aa, Artificial Sequence
  VP160
DALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDML

SEQ ID NO: 45 - protein, 7 aa, Artificial Sequence
  nuclear localization signal
PKKKRKV

(19) United States
(12) Patent Application Publication
(10) Pub. No.: US 2021/0332094 A1
(43) Pub. Date: Oct. 28, 2021

(54) NOVEL TRANSCRIPTION ACTIVATOR

(71) Applicant: MODALIS THERAPEUTICS CORPORATION, Tokyo (JP)

(72) Inventors: Tetsuya YAMAGATA, Cambridge, MA (US); Yuanbo QIN, Cambridge, MA (US)

(73) Assignee: MODALIS THERAPEUTICS CORPORATION, Tokyo (JP)

(21) Appl. No.: 17/264,594

(22) PCT Filed: Aug. 6, 2019
(86) PCT No.: PCT/JP2019/030972

§ 371 Date: Jan. 29, 2021

Related U.S. Application Data
(60) Provisional application No. 62/715,432, filed on Aug. 7, 2018.

(51) Int. Cl. C07K 14/47; C12N 9/22; C12N 7/00; C12N 15/86; C12N 15/90

(52) U.S. Cl. CPC C07K 14/4705; C12N 9/22; C12N 7/00; C12N 15/86; A61K 38/00; C07K 2319/71; C12N 2800/80; C12N 2750/14143; C12N 15/907

(57) ABSTRACT

The present invention provides a transcription activator consisting of not more than 200 amino acid sequences and containing VP64 and a transcription activation site of RTA. The present invention also provides a complex of a nucleic acid sequence-recognizing module specifically binding to a target nucleotide sequence in a double-stranded DNA and the transcription activator.

### TECHNICAL FIELD

The present invention relates to a novel transcription activator comprising VP64 and a transcription activation site of R-Trans activator (RTA). In addition, it relates to a complex of a nucleic acid sequence-recognizing module specifically binding to a target nucleotide sequence in a double-stranded DNA and the aforementioned transcription activator.

### BACKGROUND ART

In recent years, genome editing is attracting attention as a technique for modifying the object gene and genome region in various species. For example, a method of performing recombination at a targeted gene locus in DNA in a plant cell or insect cell as a host, by using a zinc finger nuclease (ZFN) wherein a zinc finger DNA binding domain and a non-specific DNA cleavage domain are linked (Patent Literature 1), and a method of cleaving or modifying a target gene in a particular nucleotide sequence or a site adjacent thereto by using TALEN wherein a transcription activator-like (TAL) effector which is a DNA binding module that the plant pathogenic bacteria Xanthomonas has, and a DNA endonuclease are linked (Patent Literature 2) have been reported. In addition, Cas9 nuclease derived from Streptococcus pyogenes is widely used as a powerful genome editing tool in eukaryotes having a repair pathway of double-stranded DNA breaks (DSB) (e.g., Patent Literature 3, Non Patent Literatures 1, 2).

Techniques for site-specific transcription regulation have also been developed by applying genomic editing techniques. For example, a method for activating or suppressing a targeted gene has been reported which includes binding ZF or TALE, or a protein or complex in which a transcription activation domain or a transcription suppressing domain (generally, VP64 is used for activation and KRAB is used for suppression) is fused with Cas9 (dCas9) system lacking the ability to cleave both strands of a double-stranded DNA to a promoter or enhancer sequence of the object gene (e.g., Non Patent Literature 3).

However, the transcription activation by using VP64 has problems in that sufficient transcription activation ability is not achieved by merely using one VP64 molecule and it is necessary to bind multiple TALE-VP64 and dCas9-VP64/sgRNA complexes to one gene (e.g., Non Patent Literature 3). To overcome this point, for example, a method using a transcription activator in which other transcription activation factors (p65 and RTA) are bound to VP64 has been reported (e.g., Non Patent Literature 4).

### CITATION LIST

### Patent Literature

PTL 1: WO 03/087341 A2

PTL 2: WO 2011/072246 A2

PTL 3: WO 2013/176772 A1

### Non Patent Literature

NPL 1: Mali P, et al., Science 339: 823-827 (2013)

NPL 2: Cong L, et al., Science 339: 819-823 (2013)

NPL 3: Hu J, et al., Nucleic Acids Res, 42: 4375-4390 (2014)

NPL 4: Chavez A, et al., Nat Methods, 12: 326-328 (2015)

### SUMMARY OF INVENTION

### Technical Problem

However, when p65 and RTA are bound to VP64, the total molecular weight thereof becomes large. Therefore, a problem occurs in that the nucleic acid encoding the complex of the CRISPR/Cas9 system and the transcription activator is under restriction in terms of size, and cannot be mounted on an adeno-associated virus (AAV) vector as an all-in-one nucleic acid. Accordingly, one of the challenges with AAV-mediated delivery is to provide a transcription activator in a size mountable on an AAV vector and capable of sufficiently exerting the transcription activation ability.

### Solution to Problem

The present inventors took note of multiple proteins having known to have transcription activation ability, and had an inventive idea that activators capable of solving the above-mentioned problem may be produced by combining such proteins appropriately. Based on the idea, they have conducted intensive studies and found that reducing the protein size and yet preserving sufficient transcription activation ability can be both achieved by combining VP64 and RTA. Based on this finding, they have conducted further studies and completed the present invention.

Therefore, the present invention provides the following.

[1] A transcription activator consisting of not more than 200 amino acids and comprising VP64 and a transcription activation site of RTA.

[2] The transcription activator of [1], wherein the aforementioned VP64 comprises

(1) the amino acid sequence shown in SEQ ID NO: 1,

(2) the amino acid sequence of (1) wherein 1 or several amino acids are deleted, substituted and/or added, or

(3) an amino acid sequence 90% or more identical to the amino acid sequence of (1).

[3] The transcription activator of [1] or [2], wherein the aforementioned transcription activation site of RTA comprises

(4) the sequence shown in SEQ ID NO: 2,

(5) the sequence shown in SEQ ID NO: 3,

(6) the amino acid sequence of (4) or (5) wherein 1 or several amino acids are deleted, substituted and/or added, or

(7) an amino acid sequence 90% or more identical to the amino acid sequence of (4) or (5).

[4] A complex comprising a nucleic acid sequence-recognizing module specifically binding to a target nucleotide sequence in a double-stranded DNA and the transcription activator of any one of [1] to [3] bonded to each other, and activating transcription of a targeted gene in the DNA.

[5] The complex of [4], wherein the aforementioned nucleic acid sequence-recognizing module comprises a CRISPR effector protein lacking the ability to cleave at least one strand of the double-stranded DNA.

[6] The complex of [5], wherein the aforementioned CRISPR effector protein lacks the ability to cleave both strands of the double-stranded DNA.

[7] The complex of [5] or [6], wherein the CRISPR effector protein is derived from Staphylococcus aureus or Campylobacter jejuni.

[8] A nucleic acid encoding the transcription activator of any one of [1] to [3].

[9] A nucleic acid encoding the complex of any one of [4] to [7].

[10] A vector comprising the nucleic acid of [8] or [9].

[11] The vector of [10], wherein the aforementioned vector is an adeno-associated virus vector.

[12] A method for activating transcription of a targeted gene in a cell, comprising a step of introducing the complex of any one of [4] to [7], the nucleic acid of [8] or [9], or the vector of [10] or [11] into the cell.

[13] The method of [12], wherein the cell is a mammalian cell.

[14] The method of [13], wherein the aforementioned mammal is a human.

### Advantageous Effects of Invention

According to the present invention, a novel transcription activator having a size mountable on an AAV vector and capable of sufficiently exerting transcription activation ability is provided. Furthermore, a complex of a nucleic acid sequence-recognizing module specifically binding to a target nucleotide sequence in a double-stranded DNA and the aforementioned transcription activator, and a method for activating transcription of a targeted gene in a cell by using the complex are provided.

### BRIEF DESCRIPTION OF DRAWINGS

FIG. 1 shows the structure of AAV vector and the ten activation moieties when dSaCas9 is used as a CRISPR effector protein. The number of bases in the Figure is indicated by the length including the stop codon.

FIG. 2 shows MYD88 gene activation by the nine activation moieties. In respective gRNAs, each bar graph shows the results of Only sgRNA, VP64, VP160, VM (VP64-MyoD), VH (VP64-HSF1), V32p65 (VP32-p65), VR (VP64-miniRTA), V64P65 (VP64-p65), VPH and VPR in this order from the left.

TABLE 1

| | sgMYD88_1 | | sgMYD88_2 | | sgMYD88_3 | |
|---|---|---|---|---|---|---|
| | (n = 3) | | (n = 3) | | (n = 3) | |
| MYD88 | Average | SD | Average | SD | Average | SD |
| Only sgRNA | 1 | NA | 1 | NA | 1 | NA |
| VP64 | 1.07 | 0.04 | 1.14 | 0.03 | 1.80 | 0.25 |
| VP160 | 1.42 | 0.27 | 1.76 | 0.15 | 2.35 | 0.21 |
| VM | 1.21 | 0.19 | 1.61 | 0.21 | 2.15 | 0.16 |
| VH | 1.04 | 0.18 | 1.55 | 0.24 | 1.84 | 0.05 |
| V32p65 | 1.20 | 0.26 | 1.90 | 0.10 | 2.31 | 0.25 |
| VR | 2.31 | 0.39 | 3.88 | 0.47 | 6.03 | 1.10 |
| V64P65 | 1.85 | 0.38 | 2.61 | 0.27 | 3.89 | 0.57 |
| VPH | 4.35 | 0.63 | 5.10 | 0.60 | 6.72 | 0.75 |
| VPR | 6.18 | 0.97 | 7.68 | 0.89 | 8.43 | 1.40 |

FIG. 3 shows FGF21 gene activation by the nine activation moieties. In respective gRNAs, each bar graph shows the results of Only sgRNA, VP64, VP160, VM (VP64-MyoD), VH (VP64-HSF1), V32p65 (VP32-p65), VR (VP64-miniRTA), V64P65 (VP64-p65), VPH and VPR in this order from the left.

TABLE 2

| | sgFGF_1 | | sgFGF_2 | | sgFGF_3 | |
|---|---|---|---|---|---|---|
| | (n = 3) | | (n = 3) | | (n = 3) | |
| FGF21 | Average | SD | Average | SD | Average | SD |
| Only sgRNA | 1 | NA | 1 | NA | 1 | NA |
| VP64 | 4.05 | 1.92 | 3.88 | 0.88 | 1.47 | 0.27 |
| VP160 | 7.08 | 0.71 | 7.56 | 0.33 | 3.98 | 1.03 |
| VM | 2.63 | 0.98 | 3.20 | 0.77 | 1.18 | 0.75 |
| VH | 4.79 | 0.89 | 8.21 | 3.17 | 1.60 | 0.42 |
| V32p65 | 4.61 | 0.93 | 6.84 | 1.80 | 0.92 | 0.31 |
| VR | 9.13 | 2.23 | 11.68 | 3.51 | 4.17 | 0.97 |
| V64P65 | 12.65 | 3.65 | 17.87 | 2.02 | 2.37 | 0.41 |
| VPH | 19.19 | 2.46 | 31.10 | 6.50 | 4.75 | 1.47 |
| VPR | 28.23 | 3.63 | 53.28 | 5.04 | 7.51 | 0.96 |

FIG. 4 shows GCG gene activation by the nine activation moieties. In respective gRNAs, each bar graph shows the results of Only sgRNA, VP64, VP160, VM (VP64-MyoD), VH (VP64-HSF1), V32p65 (VP32-p65), VR (VP64-miniRTA), V64P65 (VP64-p65), VPH and VPR in this order from the left.

TABLE 3

| | sgGCG_1 | | sgGCG_2 | | sgGCG_3 | |
|---|---|---|---|---|---|---|
| | (n = 3) | | (n = 3) | | (n = 3) | |
| GCG | Average | SD | Average | SD | Average | SD |
| Only sgRNA | 1 | NA | 1 | NA | 1 | NA |
| VP64 | 2.40 | 1.43 | 3.94 | 1.00 | 1.99 | 0.21 |
| VP160 | 54.93 | 3.34 | 25.97 | 5.64 | 6.67 | 0.51 |
| VM | 5.93 | 0.37 | 3.75 | 0.94 | 1.69 | 0.70 |
| VH | 3.73 | 1.38 | 2.82 | 0.77 | 1.98 | 0.63 |
| V32p65 | 1.99 | 0.66 | 1.99 | 1.37 | 0.96 | 0.65 |
| VR | 447.92 | 32.73 | 109.06 | 11.81 | 31.61 | 8.47 |
| V64P65 | 83.65 | 23.05 | 20.30 | 4.82 | 7.99 | 0.39 |
| VPH | 708.07 | 115.67 | 101.32 | 12.27 | 47.87 | 7.70 |
| VPR | 1274.30 | 205.93 | 328.06 | 88.78 | 125.96 | 17.78 |

FIG. 5 shows MyD88 gene activation by VP64-miniRTA and VP64-microRTA.

### DESCRIPTION OF EMBODIMENTS

As used herein, the singular forms “a”, “an” and “the” are intended to include both the singular and plural forms, unless the language explicitly indicates otherwise with words like “only” “single” and/or “one”. It will be further understood that the terms “comprises”, “comprising”, “includes” and/or “including” when used herein, specify the presence of stated features, steps, operations, elements, ideas, and/or components, but do not themselves preclude the presence or addition of one or more other features, steps, operations, elements, components, ideas, and/or groups thereof.

The present invention provides a novel transcription activator comprising VP64 and a transcription activation site of R-Trans activator (RTA) of Epstein-Ban Virus (hereinafter sometimes to be referred to as “the activator of the present invention”). Transcription of targeted gene can be activated by the transcription activator of the present invention.

In the present invention, VP64 means a peptide consisting of 4 repeats in tandem of a domain consisting of the 437th-447th amino acid residues of Herpes Simplex Virus-derived VP16 (DALDDFDLDML; SEQ ID NO: 21) with a peptide linker consisting of glycine and serine (GS) ([DALDDFDLDML]-GS-[DALDDFDLDML]-GS-[DALDDFDLDML]-GS-[DALDD FDLDML]; SEQ ID NO: 1) (Beerli R R, et al., Proc Natl Acad Sci USA. 95(25):14628-33 (1998)) or a variant thereof having a transcription activity ability. Examples of such variant include the amino acid sequence shown in SEQ ID NO: 1 wherein 1 or several (e.g., 2, 3, 4, 5 or more) amino acids are deleted, substituted and/or added. Specific examples thereof include, but are not limited to, a variant in which the linker part is substituted by other linker (e.g., a peptide linker consisting of G, S, GG, SG, GGG, GSG, GSGS (SEQ ID NO: 22), GSSG (SEQ ID NO: 23), GGGGS (SEQ ID NO: 24), GGGAR (SEQ ID NO: 25), GSGSGS (SEQ ID NO: 26) or SGQGGGGSG (SEQ ID NO: 27) and the like). Alternatively, as the aforementioned variant, a peptide consisting of an amino acid sequence not less than 90% (e.g., 90%, 91%, 92%, 93%, 94%, 95%, 96%, 97%, 98%, 99% or above) identical with the amino acid sequence shown in SEQ ID NO: 1 can be mentioned. In addition, a peptide consisting of 10 repeats in tandem of the above-mentioned domain (DALDDFDLDML; SEQ ID NO: 21) ([DALDDFDLDML]-GS-[DALDDFDLDML]-GS-[DALDDFDLDML]-GS-[DALDD FDLDML]-GS-[DALDDFDLDML]-GS-[DALDDFDLDML]-GS-[DALDDFDLDML]-GS-[DALDDFDLDML]-GS-[DALDDFDLDML]-GS-[DALDDFDLDML]; SEQ ID NO: 44) is called VP160.

RTA is a protein consisting of 605 amino acid residues and having transcription activation ability (GenBank Accession Number: CEQ33017) (SEQ ID NO: 4), and it is known that its C-terminal domain is important for transcription activation (Hardwick J M, J Virol, 66(9):5500-8, 1992). As the aforementioned domain, a region consisting of the 493rd-605th amino acid sequence of RTA (SEQ ID NO: 2) can be specifically mentioned. Among others, it is known that a region consisting of the 520th-605th amino acid sequence (SEQ ID NO: 3) is important. Therefore, RTA contained in the activator of the present invention is preferably a transcription activation site containing the amino acid sequence shown in SEQ ID NO: 2 or SEQ ID NO: 3, or a variant thereof having a transcription activation ability. Examples of such variant include the amino acid sequence shown in SEQ ID NO: 2 or 3 wherein 1 or several (e.g., 2, 3, 4, 5 or more) amino acids are deleted, substituted and/or added. Specifically, since the 564th leucine residue, the 566th leucine residue, the 570th leucine residue, the 578th leucine residue, the 581st phenylalanine residue and the 582nd leucine residue in RTA are known to be important for the transcription activation ability, a variant in which amino acid residues other than these amino acid residues are deleted, substituted and the like, and the like can be mentioned, though not limited to these modifications. Alternatively, as the aforementioned variant, a peptide consisting of an amino acid sequence not less than 90% (e.g., 90%, 91%, 92%, 93%, 94%, 95%, 96%, 97%, 98%, 99% or above) identical with the amino acid sequence shown in SEQ ID NO: 2 or 3 can be mentioned. In the present specification, a peptide consisting of the sequence shown in SEQ ID NO: 2 is sometimes referred to as “miniRTA” and a consisting of the sequence shown in SEQ ID NO: 3 is sometimes referred to as “microRTA”.

The activator of the present invention contains VP64 and a transcription activation site of RTA. VP64 and RTA may be bonded via a linker (e.g., the aforementioned peptide linker) or directly bonded without via a linker. The VP64 and a transcription activation site of RTA may be arranged in this order from the N-terminus to the C-terminus or may be arranged in reverse order. Specific examples of the activator of the present invention include the amino acid sequence shown in SEQ ID NO: 6 or 8, the amino acid sequence shown in SEQ ID NO: 6 or 8 wherein 1 or several (e.g., 2, 3, 4, 5 or more) amino acids are deleted, substituted and/or added, and an activator containing an amino acid sequence not less than 90% (e.g., 90%, 91%, 92%, 93%, 94%, 95%, 96%, 97%, 98%, 99% or above) identical with the amino acid sequence shown in SEQ ID NO: 6 or 8.

The identity of the amino acid sequence can be calculated using homology calculation algorithm NCBI BLAST (National Center for Biotechnology Information Basic Local Alignment Search Tool) (https://blast.ncbi.nlm.nih.gov/Blast.cgi) and under the following conditions (expectancy=10; gap allowed; matrix=BLOSUM62; filtering=OFF). It is understood that for determining identity a sequence of the invention over its entire length is compared to another sequence. In other words, identity according to the invention excludes comparing short fragments (e.g. 1 to 3 amino acids) of a sequence of the invention to another sequence or vice versa.

The activator of the present invention is not particularly limited as long as it can activate transcription of the targeted gene. For downsizing, it preferably consists of not more than 200 (e.g., 200, 190, 180, 170, 169, 168, 167 or more) amino acids and preferably not less than 110 (e.g., 110, 120, 130, 135, 136, 137, 138, 139, 140 or less) amino acids. In a preferable embodiment, an activator consisting of about 140 or about 167 amino acids is used.

In another embodiment, a complex in which a nucleic acid sequence-recognizing module and the activator of the present invention are bound (hereinafter sometimes to be referred to as “the complex of the present invention”) is provided.

In the present invention, the “nucleic acid sequence-recognizing module” means a molecule or molecule complex having an ability to specifically recognize and bind to a particular nucleotide sequence (i.e., target nucleotide sequence) on a DNA strand. Binding of the nucleic acid sequence-recognizing module to a target nucleotide sequence enables the activator of the present invention linked to the module to specifically act on a targeted site of a double stranded DNA.

The complex of the present invention encompasses not only one constituted of plural molecules, but also one having a nucleic acid sequence-recognizing module and the activator of the present invention in a single molecule, like a fusion protein.

A target nucleotide sequence in a double stranded DNA to be recognized by the nucleic acid sequence-recognizing module in the complex of the present invention is not particularly limited as long as the module specifically binds to, and may be any sequence in the double stranded DNA. The length of the target nucleotide sequence only needs to be sufficient for specific binding of the nucleic acid sequence-recognizing module. For example, when a mammalian genomic DNA is targeted, the sequence is, according to the genome size, preferably not less than 12 nucleotides (e.g., 12 nucleotides, 15 nucleotides, 18 nucleotides, 19 nucleotides, 20 nucleotides or more) and not more than 25 nucleotides (e.g., 25 nucleotides, 24 nucleotides, 23 nucleotides, 22 nucleotides or less).

Examples of the nucleic acid sequence-recognizing module of the complex of the present invention include, but are not limited to, a CRISPR-GNDM system in which a CRISPR effector protein lacks the ability to cleave at least one strand (preferably both strands) of an double-stranded DNA, a zinc finger motif, a TAL effector, PPR motif and the like, as well as a fragment containing a DNA binding domain of a protein capable of specifically binding to DNA such as restriction enzyme, transcription factor, RNA polymerase and the like. Preferred are CRISPR-GNDM system, zinc finger motif, TAL effector, PPR motif and the like, of which a CRISPR-GNDM system in which a CRISPR effector protein lacks the ability to cleave both strands of a double-stranded DNA is particularly preferable.

A zinc finger motif is constituted by linkage of 3-6 different Cys2His2 type zinc finger units (1 finger recognizes about 3 bases), and can recognize a target nucleotide sequence of 9-18 bases. A zinc finger motif can be produced by a known method such as Modular assembly method (Nat Biotechnol (2002) 20: 135-141), OPEN method (Mol Cell (2008) 31: 294-301), CoDA method (Nat Methods (2011) 8: 67-69), Escherichia coli one-hybrid method (Nat Biotechnol (2008) 26:695-701) and the like. The above-mentioned Patent Literature 1 can be referred to as for the detail of the zinc finger motif production.

A TAL effector has a module repeat structure with about 34 amino acids as a unit, and the 12th and 13th amino acid residues (called RVD) of one module determine the binding stability and base specificity. Since each module is highly independent, TAL effector specific to a target nucleotide sequence can be produced by simply connecting the module. For TAL effector, a production method utilizing an open resource (REAL method (Curr Protoc Mol Biol (2012) Chapter 12: Unit 12.15), FLASH method (Nat Biotechnol (2012) 30: 460-465), and Golden Gate method (Nucleic Acids Res (2011) 39: e82) etc.) have been established, and a TAL effector for a target nucleotide sequence can be designed comparatively conveniently. The above-mentioned Patent Literature 2 can be referred to as for the detail of the production of TAL effector.

PPR motif is constituted such that a particular nucleotide sequence is recognized by a continuation of PPR motifs each consisting of 35 amino acids and recognizing one nucleic acid base, and recognizes a target base only by 1, 4 and ii(-2) amino acids of each motif. Motif constitution has no dependency, and is free of interference of motifs on both sides. Therefore, like TAL effector, a PPR protein specific to the target nucleotide sequence can be produced by simply connecting PPR motifs. WO 2011/111829 A1 can be referred to as for the detail of the production of PPR motif.

When a fragment of restriction enzyme, transcription factor, RNA polymerase and the like is used, since the DNA binding domains of these proteins are well known, a fragment containing the domain and free of a DNA double strand cleavage ability can be easily designed and constructed.

As for zinc finger motif, production of many actually functionable zinc finger motifs is not easy, since production efficiency of a zinc finger that specifically binds to a target nucleotide sequence is not high and selection of a zinc finger having high binding specificity is complicated. While TAL effector and PPR motif have a high degree of freedom of target nucleic acid sequence recognition as compared to zinc finger motif, a problem remains in the efficiency since a large protein needs to be designed and constructed every time according to the target nucleotide sequence. In contrast, since the CRISPR-GNDM system recognizes the object double stranded DNA sequence by a guide nucleotide complementary to the target nucleotide sequence, any sequence can be targeted by simply synthesizing an oligonucleotide capable of specifically forming a hybrid with the target nucleotide sequence. Therefore, in a more preferable embodiment of the present invention, a CRISPR-GNDM system is used as a nucleic acid sequence-recognizing module.

When the CRISPR-GNDM system of the present invention is used, transcription of the targeted gene can be sufficiently activated by recruiting a mutant CRISPR effector protein lacking the ability to cleave at least one strand (preferably both strands) of a double-stranded DNA (hereinafter to be also simply referred to as “CRISPR effector protein”). The transcription regulatory region of the targeted gene may be any region of the gene as long as the transcription of the gene is activated by recruiting CRISPR effector protein and the activator of the present invention bonded thereto. Examples of such region include a promoter region and an enhancer region, intron, exon and the like of the targeted gene.

In the present specification, the “CRISPR-GNDM system” means a system comprising (a) a class 2 CRISPR effector protein (e.g., dCas9 or dCpf1) or a complex of said CRISPR effector protein and the activator of the present invention, and (b) a guide nucleotide (gN) that is complementary to a sequence of an transcription regulatory region of a target gene, which allows recruiting the CRISPR effector protein and the transcription regulator bound therewith to the transcription regulatory region of the target gene. Using the aforementioned system, transcription activation of the gene becomes possible via the activator of the present invention bonded to the CRISPR effector protein.

The “CRISPR effector protein” to be used in the present invention is not particularly limited as long as it forms a complex with gN, recognizes and binds the target nucleotide sequence in the object gene and the protospacer adjacent motif (PAM) adjacent thereto. Preferred is Cas9 or Cpf1 or a variant thereof. Examples of the Cas9 include, but are not limited to, Streptococcus pyogene-derived Cas9 (SpCas9; PAM sequence NGG (N is A, G, T or C, hereinafter the same), Streptococcus thermophilus-derived Cas9 (StCas9; PAM sequence NNAGAAW), Neisseria meningitidis-derived Cas9 (NmCas9; PAM sequence NNNNGATT), Staphylococcus aureus-derived Cas9 (SaCas9; PAM sequence: NNGRRT), Campylobacter jejuni-derived Cas9 (CjCas9; PAM sequence: NNNVRYM (V is A, G or C; R is A or G; Y is T or C; M is A or C)). In view of the size, Cas9 is preferably SaCas9 or CjCas9 or a variant thereof. Examples of the Cpf1 include, but are not limited to, Francisella novicida-derived Cpf1 (FnCpf1; PAM sequence NTT), Acidaminococcus sp.-derived Cpf1 (AsCpf1; PAM sequence NTTT), Lachnospiraceae bacterium-derived Cpf1 (LbCpf1; PAM sequence NTTT) and the like. As the CRISPR effector protein to be used in the present invention, the protein in which the ability of CRISPR effector protein to cleave at least one strand (preferably both strands) of the double-stranded DNA is inactivated is used. For example, in the case of SpCas9, a variant in which the 10th Asp residue is converted to the Ala residue and/or the 840th His residue is converted to the Ala residue (variant lacking the ability to cleave both strands of a double-stranded DNA is sometimes referred to as “dSpCas9”) can be used. Alternatively, in the case of SaCas9, a variant in which the 10th Asp residue is converted to the Ala residue and/or the 556th Asp residue, the 557th His residue and/or the 580th Asn residue are/is converted to the Ala residue (variant lacking the ability to cleave both strands of a double-stranded DNA is sometimes referred to as “dSaCas9”) can be used. In the case of CjCas9, a variant in which the 8th Asp residue is converted to the Ala residue and/or the 559th His residue is converted to the Ala residue (variant lacking the ability to cleave both strands of a double-stranded DNA is sometimes referred to as “dCjCas9”) can be used. In the case of FnCpf1, a variant in which the 917th Asp residue is converted to the Ala residue and/or the 1006th Glu residue is converted to the Ala residue can be used. Furthermore, as long as the binding ability to the target nucleotide sequence can be maintained, a variant in which a part of the amino acids of these proteins is modified may also be used. Examples of the variant include a shortened variant in which a part of the amino acid sequence is deleted. Examples of such variant specifically include dSaCas9 in which the 721st-the 745th amino acids are deleted (the deleted part may be substituted by the above-described peptide linker and the like) and the like.

The second element of the CRISPR-GNDM system of the present invention is a guide nucleotide (gN) that contains a nucleotide sequence (hereinafter also referred to as “targeting sequence”) complementary to the nucleotide sequence adjacent to PAM of the targeted strand in the transcription regulatory region of the targeted gene. When the CRISPR effector protein is dCas9, the gN is provided as a chimeric nucleotide of truncated crRNA and tracrRNA (i.e., single guide RNA (sgRNA)), or combination of separate crRNA and tracrRNA. The gN may be provided in a form of RNA, DNA or DNA/RNA chimera. Thus, hereinafter, as long as technically possible, the terms “sgRNA”, “crRNA” and “tracrRNA” are used to also include the corresponding DNA and DNA/RNA chimera in the context of the present invention.

The “targeted strand” here means a strand forming a hybrid with crRNA of the target nucleotide sequence, and an opposite strand thereof that becomes single-stranded by hybridization to the targeted strand and crRNA is referred to as a “non-targeted strand”. When the target nucleotide sequence is to be expressed by one of the strands (e.g., when PAM sequence is indicated, when positional relationship of target nucleotide sequence and PAM is shown etc.), it is represented by a sequence of the non-targeted strand.

The targeting sequence is not limited as long as it can specifically hybridize with the targeted strand at a transcription regulatory region of a targeted gene and recruit the CRISPR effector protein and the activator of the present invention bound therewith to the transcription regulatory region. For example, when dSaCas9 is used as the CRISPR effector protein, the targeting sequences listed in Table 1 are exemplified. In Table 1, while targeting sequences consisting of 21 nucleotides are described, the length of the targeting sequence is preferably not less than 12 nucleotides (e.g., 12 nucleotides, 15 nucleotides, 18 nucleotides, 19 nucleotides, 20 nucleotides or more), and not more than 25 nucleotides (e.g., 25 nucleotides, 24 nucleotides, 23 nucleotides, 22 nucleotides or less). In a preferable embodiment, it is 21 nucleotides.

When Cas9 is used as the CRISPR effector protein, the targeting sequence can be designed, for example, using a guide nucleotide design website open to public (CRISPR Design Tool, CRISPRdirect etc.) by listing up 21 mer sequences having PAM (e.g., NNGRRT for SaCas9) adjacent to the 3′-side from the CDS sequences of the object gene. A candidate sequence having a small number of off-target sites in the host genome can be used as a targeting sequence. When the guide nucleotide design software to be used does not have the function of searching the off-target site of the host genome, the off-target site can be searched by, for example, subjecting the host genome to Blast search on 8 to 12 nucleotides (seed sequence with high discrimination ability of the target nucleotide sequence) on the 3′ side of the candidate sequence. Even when a CRISPR effector protein recognizing a different PAM is used, the targeting sequence can be designed and produced by a similar method. Unless otherwise specified, in the present specification, the targeting sequence is shown as a DNA sequence. When an RNA is used as the gN, “T” should be read as “U” in each sequence.

TABLE 4

| SEQ ID | Targeted | | |
|---|---|---|---|
| NO | gene | name | Sequence |
| 35 | MYD88 | MYD88-1 | GGTTCATACGGTCCTGCCCTC |
| 36 | MYD88 | MYD88-2 | GGAGCCACAGTTCTTCCACGG |
| 37 | MYD88 | MYD88-3 | CTCTACCCTTGAGGTCTCGAG |
| 38 | FGF21 | FGF21-1 | TGCCAGATTCCAGTTGTCCAG |
| 39 | FGF21 | FGF21-2 | ACATTCCTGAGTCTCAGAGAG |
| 40 | FGF21 | FGF21-3 | GGCTAATTTCCTGGAGCCCCT |
| 41 | GCG | GCG-1 | CTGTGAGGCTAAACAGAGCTG |
| 42 | GCG | GCG-2 | GTCTCTCACCCAATATAAGCA |
| 43 | GCG | GCG-3 | AAATCACTTAAGTTCTCTAAA |

Any of the above-mentioned nucleic acid sequence-recognizing module can be provided as a fusion protein with the above-mentioned activator of the present invention, or a protein binding domain such as SH3 domain, PDZ domain, GK domain, GB domain and the like and a binding partner thereof may be fused with a nucleic acid sequence-recognizing module and the activator of the present invention, respectively, and provided as a protein complex via an interaction of the domain and a binding partner thereof. Alternatively, a nucleic acid sequence-recognizing module and the activator of the present invention may be each fused with intein, and they can be linked by ligation after protein synthesis.

The complex of the present invention containing a complex (including fusion protein) wherein a nucleic acid sequence-recognizing module and the activator of the present invention are bonded may be contacted with a double stranded DNA as an enzyme reaction in a cell-free system. In view of the main object of the present invention, a nucleic acid encoding said complex is desirably introduced into a cell having the object double stranded DNA (e.g., genomic DNA). Therefore, the nucleic acid sequence-recognizing module and the activator of the present invention are preferably prepared as a nucleic acid encoding a fusion protein thereof, or in a form capable of forming a complex in a host cell after translation into a protein by utilizing a binding domain, intein and the like, or as a nucleic acid encoding each of them. The nucleic acid here may be a DNA or an RNA. When it is a DNA, it is preferably a double stranded DNA, and provided in the form of an expression vector disposed under regulation of a functional promoter in a host cell. When it is an RNA, it is preferably a single strand RNA.

Since the complex of the present invention wherein a nucleic acid sequence-recognizing module and the activator of the present invention are bonded does not accompany double-stranded DNA breaks (DSB), a method using the complex of the present invention can be applied to a wide range of biological materials. Therefore, the cells to be introduced with nucleic acid encoding nucleic acid sequence-recognizing module and/or the activator of the present invention can encompass cells of any species, from bacterium of Escherichia coli and the like which are prokaryotes, cells of microorganism such as yeast and the like which are lower eucaryotes, to cells of vertebrata including mammals such as human and the like, and cells of higher eukaryote such as insect, plant and the like.

A DNA encoding a nucleic acid sequence-recognizing module such as zinc finger motif, TAL effector, PPR motif, CRISPR-GNDM system and the like can be obtained by any method mentioned above for each module. A DNA encoding a sequence-recognizing module of restriction enzyme, transcription factor, RNA polymerase and the like can be cloned by, for example, synthesizing an oligoDNA primer covering a region encoding a desired part of the protein (part containing DNA binding domain) based on the cDNA sequence information thereof, and amplifying by the RT-PCR method using, as a template, the total RNA or mRNA fraction prepared from the protein-producing cells.

A mutant CRISPR effector protein can be obtained by introducing, into DNA encoding cloned CRISPR effector protein, a mutation that converts the amino acid residue at the site important for DNA cleavage activity (e.g., 10th Asp residue and 840th His residue for SpCas9, 10th Asp residue, 556th Asp residue, 557th His residue, 580th Asn residue for SaCas9, 8th ASP residue, 559th His residue for CjCas9, 917th Asp residue and 1006th Glu residue for FnCpf1 and the like, though not limited thereto) to other amino acid.

The cloned DNA may be directly, or after digestion with a restriction enzyme when desired, or after addition of a suitable linker (e.g., the above-mentioned peptide linker etc.), tag (e.g., HA tag, myc tag, MBP tag, FLAG tag etc.) and/or a nuclear localization signal (each oraganelle transfer signal when the object double stranded DNA is mitochondria or chloroplast DNA), ligated with a DNA encoding a nucleic acid sequence-recognizing module to prepare a DNA encoding a fusion protein. Alternatively, a DNA encoding a nucleic acid sequence-recognizing module, and a DNA encoding the activator of the present invention may be each fused with a DNA encoding a binding domain or a binding partner thereof, or both DNAs may be fused with a DNA encoding a separation intein, whereby the nucleic acid sequence-recognizing conversion module and the activator of the present invention are translated in a host cell to form a complex. In these cases, a linker and/or a nuclear localization signal can be linked to a suitable position of one of or both DNAs when desired. When the complex of the present invention is expressed as a fusion protein, the activator of the present invention may be fused with any of the N-terminal and the C-terminal of the nucleic acid sequence-recognizing module or a constituent component thereof (e.g., CRISPR effector protein).

A DNA encoding a nucleic acid sequence-recognizing module and/or the activator of the present invention can be obtained by chemically synthesizing the DNA strand, or by connecting synthesized partly overlapping oligoDNA short strands by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (http://www.kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

RNA encoding the nucleic acid sequence-recognizing module and/or the activator of the present invention can be prepared by, for example, preparing a vector containing a DNA encoding the module and/or the activator and transcribing same into mRNA by a known in vitro transcription system using the vector as a template. Alternatively, RNA can also be synthesized chemically.

An expression vector containing a DNA encoding the activator of the present invention or the complex of the present invention can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as λphage and the like; insect virus vectors such as baculovirus and the like (e.g., BmNPV, AcNPV); animal virus vectors such as retrovirus, vaccinia virus, adenovirus, adeno-associated virus (AAV) and the like, and the like are used. In consideration of the use in gene therapy, AAV vector is preferably used since it can express transgene for a long term and it is safe due to its derivation from a nonpathogenic virus.

The AAV vector is not particularly limited as long as the titer and infection efficiency are sufficiently secured. It is preferably not more than about 5 kb (e.g., about 5 kb, about 4.95 kb, about 4.90 kb, about 4.85 kb, about 4.80 kb, about 4.75 kb, about 4.70 kb or below). The amino acid length of the activator of the present invention is preferably not more than 200 amino acids. Thus, the total base length of the nucleic acid encoding the complex of the present invention and the nucleic acid encoding the guide nucleotide can be easily designed to be below this size limit. Therefore, the activator of the present invention has an advantage that mounting of the nucleic acid encoding the complex of the present invention and the nucleic acid encoding the guide nucleotide on separate AAV vectors is not necessary.

When a virus vector is used as an expression vector, a vector derived from a serotype suitable for infection to the object tissue or organ is preferably used. Taking AAV vector as an example, it is preferable to use a vector based on AAV 1, 2, 3, 4, 5, 7, 8, 9 or 10 when the central nervous system or retina is the target, a vector based on AAV 1, 3, 4, 6 or 9 when the heart is the target, a vector based on AAV 1, 5, 6, 9 or 10 when the lung is the target, a vector based on AAV 2, 3, 6, 7, 8, or 9 when the liver is the target, and a vector based on AAV 1, 2, 6, 7, 8, 9 when the skeletal muscle is the target. For cancer treatment, AAV 2 is preferably used. As for the serotype of AAV, for example, WO 2005/033321 A2 and the like can be referred to.

An RNA encoding a nucleic acid sequence-recognizing module and/or the activator of the present invention can be introduced into a host cell by microinjection method, lipofection method and the like. RNA introduction can be performed once or repeated multiple times (e.g., 2-5 times) at suitable intervals.

In addition, multiple DNA regions at completely different sites may be the target. Therefore, in one embodiment of the present invention, two or more kinds of nucleic acid sequence-recognizing modules that specifically bind to different target nucleotide sequences (which may be present in one object gene, or two or more different object genes, which object genes may be present on the same chromosome or different chromosomes) can be used. In this case, each one of these nucleic acid sequence-recognizing modules and the activator of the present invention form a complex. Here, a common activator of the present invention can be used. For example, when CRISPR-GNDM system is used as a nucleic acid sequence-recognizing module, a common complex of a CRISPR effector protein and the activator of the present invention (including fusion protein) is used, and two or more crRNAs, or two or more kinds of chimeric RNAs of tracrRNA and each of two or more crRNAs that respectively form a complementary strand with a different target nucleotide sequence are produced and used as gNs. On the other hand, when zinc finger motif, TAL effector and the like are used as nucleic acid sequence-recognizing modules, for example, the activator of the present invention can be fused with a nucleic acid sequence-recognizing module that specifically binds to a different target nucleotide.

A DNA encoding a gN can be chemically synthesized using a DNA/RNA synthesizer based on its sequence information. For example, a DNA encoding an gN for SaCas9 has a deoxyribonucleotide sequence encoding a crRNA containing a targeting sequence complementary to a transcription regulatory region of a targeted gene and at least a part of the “repeat” region (e.g., GUUUUAGUACUCUG; SEQ ID NO:31) of the native SacrRNA, and a deoxyribonucleotide sequence encoding tracrRNA having at least a part of the “anti-repeat” region (e.g., CAGAAUCUACUAAAAC; SEQ ID NO:32) complementary to the repeat region of the crRNA and the subsequent stem- loop 1, linker and stemloop 2 regions (AAGGCAAAAUGCCGUGUUUAUCACGUCAACUUGUUGGCGAGAUUUUUU U; SEQ ID NO:33) of the native SatracrRNA, optionally linked via a tetraloop (e.g., GAAA). On the other hand, a DNA encoding an gRNA for dCpf 1 has a deoxyribonucleotide sequence encoding a crRNA alone, which contains a targeting sequence complementary to a transcription regulatory region of a targeted gene and the preceding 5′-handle (e.g., AAUUUCUACUCUUGUAGAU; SEQ ID NO:34). When a protein other than SaCas9 and Cpf1 is used as a CRISPR effector protein, a tracrRNA for the protein to be used can be designed appropriately based on a known sequence and the like. The DNA encoding the CRISPR effector protein ligated with the DNA encoding the activator of the present invention can be subcloned into an expression vector such that said DNAs are located under the control of a promoter that is functional in a host cell of interest.

A DNA encoding gN (e.g., crRNA or crRNA-tracrRNA chimera) can be introduced into a host cell by a method similar to those described above depending on the host.

Alternatively, an RNA can be used instead of the DNA to deliver CRISPR effector molecule. In one embodiment, the CRISPR-GNDM system of the present invention comprising (a) the complex of the present invention, and (b) a gN containing a targeting sequence can be introduce into target cells or organisms in the form of RNAs encoding (a) and (b) above.

For example, the aforementioned RNA encoding the effector molecules above can be generated via in vitro transcription, and the generated mRNA can be purified for in vivo delivery. Briefly, a DNA fragment containing the CDS region of the effector molecules can be cloned down-stream of an artificial promoter from bacteriophage driving in vitro transcription (e.g. T7 T3 or SP6 promoter). The RNA can be transcribed from the promoter by adding components required for in vitro transcription such as T7 polymerase, NTPs, and IVT buffers. If need be, the RNA can be modified to reduce immune stimulation, enhance translation and nuclease stability (e.g. 5mCAP (m7G(5′)ppp(5′)G capping, ARCA; anti-Reverse Cap Analogs (3′ O-Me-M7G(5′)ppp(5′)G), 5-methylcytidine and pseudouridine modifications, 3′ poly A tail).

Alternatively, a complex of an effector protein and a gN, hereafter termed nucleoprotein (NP) (e.g., deoxyribonucleoprotein (DNP), ribonucleoprotein (RNP)), can be used to deliver CRISPR effector molecule and gN. Briefly, in vitro generated CRISPR effector protein and in vitro transcribed or chemically synthesized gN are mixed at appropriate ratios, and then encapsulated into Lipid nanoparticles (LNPs). The encapsulated LNPs can be delivered into an animal suffering from a disease or patient, and the NP complex can be delivered directly into target cells or organs.

A CRISPR effector protein can be expressed in bacteria and can be purified via affinity column. Bacteria codon-optimized cDNA sequence of the CRISPR effector protein can be cloned into bacteria expression plasmids such as pE-SUMO vector from LifeSensors. The cDNA fragment can be tagged with a small peptide sequence such as HA, 6xHis, Myc, or FLAG peptides, either on N- or C-terminal. The plasmids can be introduced into protein-expressing bacterial strains such as E. coli B834 (DE3). After induction, the protein can be purified using affinity column binding to the small peptide tag sequences, such as Ni-NTA column or anti-FLAG affinity column. The attached tag peptide can be removed by TEV protease treatment. The protein can be further purified by chromatography on a HiLoad Superdex 200 16/60 column (GE Health-care).

Alternatively, the CRISPR effector protein can be expressed in mammalian cell lines such as CHO, COS, HEK293, and Hela cell. For example, human codon-optimized cDNA sequence of the CRISPR protein can be cloned into mammalian expression plasmids (e.g., pA1-11, pXT1, pRc/CMV, pRc/RSV, pcDNAI/Neo, pSRa); vectors derived from animal virus such as retrovirus, vaccinia virus, adenovirus, adeno-associated virus, etc, and the like can be used. The cDNA fragments can be tagged with a small peptide sequence such as HA, 6xHis, Myc, or FLAG peptide, either on N- or C-terminal. The plasmids can be introduced into the protein-expressing mammalian cell lines. 2-3 days after the transfection, the transfected cells can be harvested and the expressed CRISPR protein can be purified using affinity column binding to the small peptide tag sequences said above.

The activator of the present invention can also be obtained by a method similar to the above-mentioned method.

### EXAMPLES

The invention will be more fully understood by reference to the following examples, which provide illustrative non-limiting embodiments of the invention.

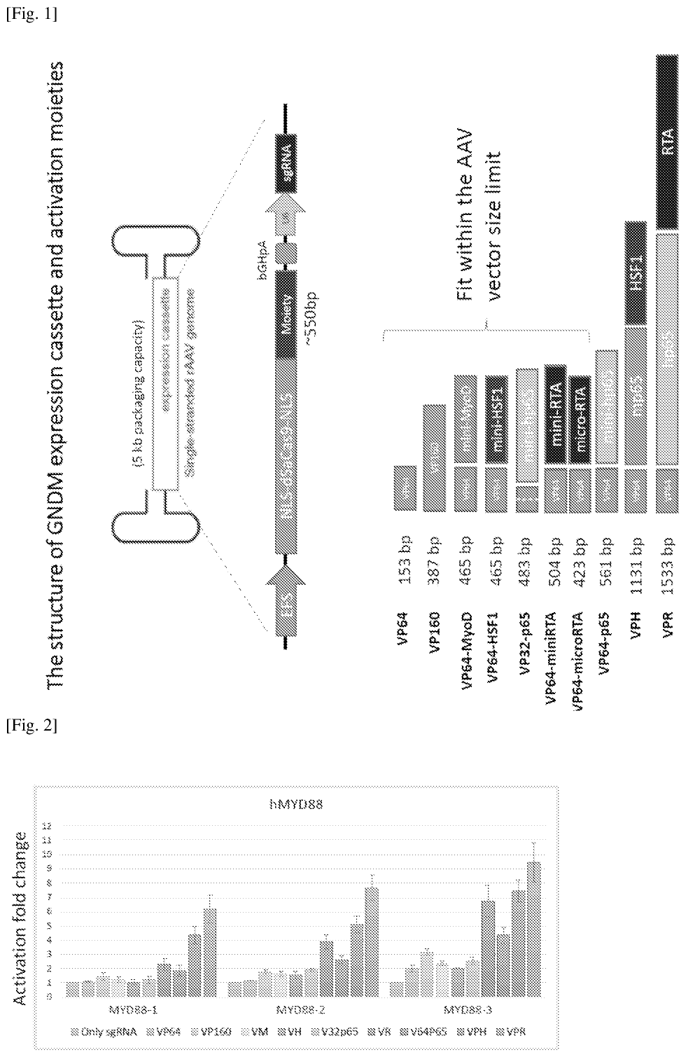

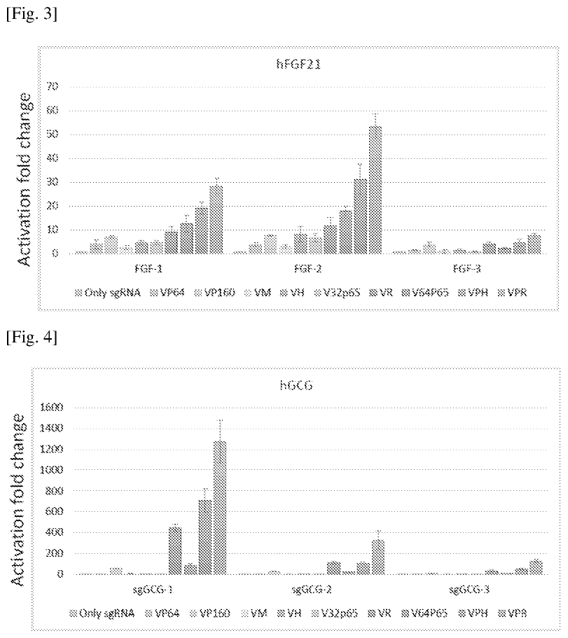

We designed and constructed new activation moieties that are small enough to fuse with dSaCas9 and fit into the AAV vector size limit of 5 kb while harboring comparable or even better transcription activating potency than existing activation moieties (FIG. 1). The existing activation moieties include VP64 (50 a.a.), VP160 (130 a.a.), VPR (520 a.a.), and P300 (617 a.a.) (described in PMID:27214048/25730490). Of these activation moieties, only VP64 and VP160 satisfy the size limit of AAV vector when fused with dSaCas9.

Therefore, we designed, constructed and tested the following seven new activation moieties fused with dSaCas9, and compared their transactivation potency with the existing three moieties (VP64, VP160 and VPR).

Amino acid and nucleotide sequence of the generated activation moieties

1. VP64-miniMYOD (154 a.a.) consists of VP64 (italics) and 1-100 a.a. from
human MYOD1 (boldface, PMID: 9710631) which are connected by a G-S-G-S
linker (underline);
(SEQ ID NO: 10)
DALDDFDLDMLGSDALDDFDLDMLGSDALDDFDLDMLGSDALDDFDLDMLGSGSMELLSPPLR
DVDLTAPDGSLCSFATTDDFYDDPCFDSPDLRFFEDLDPRLMRVGALLKPEEHSHFPAAVHPA
PGAREDEHVRAPSGHHQAGRCLLWACKA
(SEQ ID NO: 9)
gatgctttagacgattttgacttagatatgcttggttcagacgcgttagacgacttcgacctagacatgttaggctcagatgca
ttggacgacttcgatttagatatgttgggctccgatgccctagatgactttgatttggatatgctaggatctggtagcatggagct
actgtcgccaccgctccgcgacgtagacctgacggcccccgacggctctctctgctcctttgccacaacggacgacttctat
gacgacccgtgtttcgactccccggacctgcgcttcttcgaggacctggacccgcgcctgatgcacgtgggcgcgctcctg
aaacccgaagagcactcgcacttccctgcggctgttcacccggcaccgggggcacgcgaggacgaacatgtcagggctc
ccagcggtcatcaccaggctggtcggtgtctgttgtgggcctgcaaggcg
2. VP64-miniHSF1 (154 a.a.) consists of VP64 (italics) and 430-529 a.a. from
human HSF1(boldface, PMID:7760831) which are connected by a G-S-S-G linker
(underline);
(SEQ ID NO: 12)
DALDDFDLDMLGSDALDDFDLDMLGSDALDDFDLDMLGSDALDDFDLDMLGSSGPDLDSSLAS
IQELLSPQEPPRPPEAENSSPDSGKQLVHYTAQPLFLLDPGSVDTGSNDLPVLFELGEGSYKS
EGDGFAEDPTISLLTGSEPPKAKDPTVS
(SEQ ID NO: 11)
gatgctttagacgattttgacttagatatgcttggttcagacgcgttagacgacttcgacctagacatgttaggctcagatgca
ttggacgacttcgatttagatatgttgggctccgatgccctagatgactttgatttggatatgctaggtagcagtgggcctgacct
tgacagcagcctggccagtatccaagagctcctgtctccccaggagccccccaggcctcccgaggcagagaacagcagc
ccggattcagggaagcagctggtgcactacacagcgcagccgctgttcctgctggaccccggctccgtggacaccggga
gcaacgacctgccggtgctgtttgagctgggagagggctcctacttctccgaaggggacggcttcgccgaggaccccacc
atctccctgctgacaggctcggagcctcccaaagccaaggaccccactgtctcc
3. VP32-miniP65 (160 a.a.) consists of VP32 (italics) and 415-546 a.a. from human
P65 (boldface, PMID:1732726) which are connected by a G-S-G-S linker (underline);
(SEQ ID NO: 14)
DALDDFDLDMLGSDALDDFDLDMLGSGSPGPPQAVAPPAPKPTQAGEGTLSEALLQLQFDDED
LGALLGNSTDPAVFTDLASVDNSEFQQLLNQGIPVAPHTTEPMLMEYPEAITRLVTGAQRPPD
PAPAPLGAPGLPNGLLSGDEDFSSIADMDSALL
(SEQ ID NO: 13)
gatgcattggacgacttcgatttagatatgttgggctccgatgccctagatgactttgatttggatatgctaggatctggtagc
cctggacctccacaggctgtggctccaccagcccctaaacctacacaggccggcgagggcacactgtctgaagctctgctg
cagctgcagttcgacgacgaggatctgggagccctgctgggaaacagcaccgatcctgccgtgttcaccgacctggccag
cgtggacaacagcgagttccagcagctgctgaaccagggcatccctgtggcccctcacaccaccgagcccatgctgatgg
aataccccgaggccatcacccggctcgtgacaggcgctcagaggcctcctgatccagctcctgcccctctgggagcacca
ggcctgcctaatggactgctgtctggcgacgaggacttcagctctatcgccgatatggatttctcagccttgctg
4. VP64-miniRTA (167 a.a.) consists of VP64 (italics) and 493-605 a.a. from
Epstein-Barr virus Replication and transcription activator (boldface, RTA;
PMID:1323708) which are connected by a G-S-G-S linker (underline);
(SEQ ID NO: 6)
DALDDFDLDMLGSDALDDFDLDMLGSDALDDFDLDMLGSDALDDFDLDMLGSGSPAPAVTPEA
SHLLEDPDEETSQAVKALREMADTVIPQKEEAAICGQMDLSHPPPRGHLDELTTTLESMTEDL
NLDSPLTPELNEILDTFLNDECLLHAMHISTGLSIFDTSLF
(SEQ ID NO: 5)
gatgctttagacgattttgacttagatatgcttggttcagacgcgttagacgacttcgacctagacatgttaggctcagatgca
ttggacgacttcgatttagatatgttgggctccgatgccctagatgactttgatttggatatgctaggatctggtagcccagcgc
ccgcagtgactcccgaggccagtcacctgttggaagatcccgatgaagagaccagccaggctgtcaaagcccttcgggag
atggccgatactgtgattccccagaaggaagaggctgcaatctgtggccaaatggacctttcccatccgcccccaaggggc
catctggatgagctgacaaccacacttgagtccatgaccgaggatctgaacctggactcacccctgaccccggaattgaacg
agattctggataccttcctgaacgacgagtgcctcttgcatgccatgcatatcagcacaggactgtccatcttcgacacatctct
gttt
5. VP64-miniP65 (186 a.a.) consists VP64 (italics) and 415-546 a.a. from human
P65 (boldface, PMID:1732726) which are connected by a G-S-G-S linker (underline);
(SEQ ID NO: 16)
DALDDFDLDMLGSDALDDFDLDMLGSDALDDFDLDMLGSDALDDFDLDMLGSGSPGPPQAVAP
PAPKPTQAGEGTLSEALLQLQFDDEDLGALLGNSTDPAVFTDLASVDNSEFQQLLNQGIPVAP
HTTEPMLMEYPEAITRLVTGAQRPPDPAPAPLGAPGLPNGLLSGDEDFSSIADMDFSALL
(SEQ ID NO: 15)
gatgctttagacgattttgacttagatatgcttggttcagacgcgttagacgacttcgacctagacatgttaggctcagatgca
ttggacgacttcgatttagatatgttgggctccgatgccctagatgactttgatttggatatgctaggatctggtagccctggacc
tccacaggctgtggctccaccagcccctaaacctacacaggccggcgagggcacactgtctgaagctctgctgcagctgca
gttcgacgacgaggatctgggagccctgctgggaaacagcaccgatcctgccgtgttcaccgacctggccagcgtggaca
acagcgagttccagcagctgctgaaccagggcatccctgtggcccctcacaccaccgagcccatgctgatggaatacccc
gaggccatcacccggctcgtgacaggcgctcagaggcctcctgatccagctcctgcccctctgggagcaccaggcctgcc
taatggactgctgtctggcgacgaggacttcagctctatcgccgatatggatttctcagccttgctg
6. VPH (376 a.a.) consists of VP64 (italics), 369-549 a.a. from murine P65
(boldface) and 407-529 a.a. from human HSF1 (underlined boldface), PMID:
25494202) which are connected by NLS (PKKKRKV)(SEQ ID NO: 45) and/or S-
G-Q-G-G-G-G-S-G linker (underline);
(SEQ ID NO: 13)
DALDDFDLDMLGSDALDDFDLDMLGSDALDDFDLDMLGSDALDDFDLDMLSSGSPKKKRKVGS
PSGQISNQALALAPSSAPVLAQTMVPSSAMVPLAQPPAPAPVLTPGPPQSLSAPVPKSTQAGE
GTLSEALLHLQFDADEDLGALLGNSTDPGVFTDLASVDNSEFQQLLNQGVSMSHSTAEPMLME
YPEAITRLVTGSQRPPDPAPTPLGTSGLENGLSGDEDFSSIADMDFSALLSQISSSGQGGGGS
GFSVDTSALLDLFSPSVTVPDMSLPDLDSSLASIQELLSPQEPPRPPEAENSSPDSGKQLVHY
TAQPLFLLDPGSVDTGSNDLPVLFELGEGSYFSEGDGFAEDPTISLLTGSEPPKAKDPTVS
(SEQ ID NO: 17)
gatgctttagacgattttgacttagatatgcttggttcagacgcgttagacgacttcgacctagacatgttaggctcagatgca
ttggacgacttcgatttagatatgttgggctccgatgccctagatgactttgatttggatatgctaagttccggatctccgaaaaa
gaaacgcaaagttggtagcccttcagggcagatcagcaaccaggccctggctctggcccctagctccgctccagtgctggc
ccagactatggtgccctctagtgctatggtgcctctggcccagccacctgctccagcccctgtgctgaccccaggaccaccc
cagtcactgagcgctccagtgcccaagtctacacaggccggcgaggggactctgagtgaagctctgctgcacctgcagttc
gacgctgatgaggacctgggagctctgctggggaacagcaccgatcccggagtgttcacagacctggcctccgtggacaa
ctctgagtttcagcagctgctgaatcagggcgtgtccatgtctcatagtacagccgaaccaatgctgatggagtaccccgaag
ccattacccggctggtgaccggcagccagcggccccccgaccccgctccaactcccctgggaaccagcggcctgcctaat
gggctgtccggagatgaagacttctcaagcatcgctgatatggactttagtgccctgctgtcacagatttcctctagtgggcag
ggaggaggtggaagcggcttcagcgtggacaccagtgccctgctggacctgttcagcccctcggtgaccgtgcccgacat
gagcctgcctgaccttgacagcagcctggccagtatccaagagctcctgtctccccaggagccccccaggcctcccgagg
cagagaacagcagcccggattcagggaagcagctggtgcactacacagcgcagccgctgttcctgctggaccccggctc
cgtggacaccgggagcaacgacctgccggtgctgtttgagctgggagagggctcctacttctccgaaggggacggcttcg
ccgaggaccccaccatctccctgctgacaggctcggagcctcccaaagccaaggaccccactgtctcc
7. VPR (510 a.a.) consists of VP64 (italics), 284-543 a.a. from human P65 (boldface,
PMID: 5970) and 416-605 a.a. from Epstein-Barr virus Replication and transcription
activator (underlined boldface, RTA; PMID:1323708) which are connected by NLS
(PKKKRKV) and/or G-S-G-S-G-S linker (underline)
(SEQ ID NO: 20)
DALDDFDLDMLGSDALDDFDLDMLGSDALDDFDLDMLINSRSSGSPKKKRKVGSQYLPDTDDR
HRIEEKRKRTYETFKSIMKKSPFSGPTDPRPPPRRIAVPSRSSASVPKPAPQPYPFTSSLSTI
NYDEFFTMVFPSGQISQASALAPAPPQVLPQAPAPARAPAMVSALAQAPAPVPVLAPGPPQAV
APPAPKPTQAGEGTLSEALLQLQFDDEDLGALLGNSTDPAVFTDLASVDNSEFQQLLNQGIPV
APHTTEPMLMEYPEAITRLVTGAQRPPDPAPAPLGAPGLPNGLLSGDEDFSSIADMDFSALLG
SGSGSRDSREGMFLPKPEAGSAISDVFEGREVCQPKRIRPFHPPGSPWANRPLPASLAPTPTG
PVHEPVGSLTPAPVPQPLDPAPAVTPEASHLLEDPDEETSQAVKALREMADTVIPQKEEAAIC
GQMDLSHPPPRGHLDELTTTLESMTEDLNLDSPLTPELNEILDTFLNDECLLHAMHISTGLSI
FDTSLF
(SEQ ID NO: 19)
gacgccctcgatgattttgaccttgacatgcttggttcggatgcccttgatgactttgacctcgacatgctcggcagtgacgccc
ttgatgatttcgacctggacatgctgattaactctAgaagttccggatctccgaaaaagaaacgcaaagttggtagccagtac
ctgcccgacaccgacgaccggcaccggatcgaggaaaagcggaagcggacctacgagacattcaagagCatcatgaag
aagtcccccttcagcggccccaccgaccctagacctccacctagaagaatcgccgtgcccagcagatccagcgccagcgt
gccaaaacctgccccccagccttaCcccttcaccagcagcctgagcaccatcaactacgacgagttccctaccatggtgttc
cccagcggccagatctctcaggcctctgctctggctccagcccctcctcaggtgctgcctcaggctcctgctcctgcaccag
ctccagccatggtgtctgcactggctcaggcaccagcacccgtgcctgtgctggctcctggacctccacaggctgtggctcc
accagcccctaaacctacacaggccggcgagggcacactgtctgaagctctgctgcagctgcagttcgacgacgaggatc
tgggagccctgctgggaaacagcaccgatcctgccgtgttcaccgacctggccagcgtggacaacagcgagttccagcag
ctgctgaaccagggcatccctgtggcccctcacaccaccgagcccatgctgatggaataccccgaggccatcacccggct
cgtgacaggcgctcagaggcctcctgatccagctcctgcccctctgggagcaccaggcctgcctaatggactgctgtctgg
cgacgaggacttcagctctatcgccgatatggatttctcagccttgctgggctctggcagcggcagccgggattccagggaa
gggatgtttttgccgaagcctgaggccggctccgctattagtgacgtgtttgagggccgcgaggtgtgccagccaaaacga
atccggccatttcatcctccaggaagtccatgggccaaccgcccactccccgccagcctcgcaccaacaccaaccggtcca
gtacatgagccagtcgggtcactgaccccggcaccagtccctcagccactggatccagcgcccgcagtgactcccgaggc
cagtcacctgttggaggatcccgatgaagagacgagccaggctgtcaaagcccttcgggagatggccgatactgtgattcc
ccagaaggaagaggctgcaatctgtggccaaatggacctttcccatccgcccccaaggggccatctggatgagctgacaa
ccacacttgagtccatgaccgaggatctgaacctggactcacccctgaccccggaattgaacgagattctggataccttcctg
aacgacgagtgcctcttgcatgccatgcatatcagcacaggactgtccatcttcgacacatctctgttt
8. VP64-microRTA (140 a.a.) consists of VP64 (italics) and 520-605 a.a. from
Epstein-Barr virus Replication and transcription activator (boldface, RTA;
PMID:1323708) which are connected by a G-S-G-S linker (underline);
(SEQ ID NO: 8)
DALDDFDLDMLGSDALDDFDLDMLGSDALDDFDLDMLGSDALDDFDLDMLGSGSREMADTVIP
QKEEAAICGQMDLSHPPPRGHLDELTTTLESMTEDLNLDSPLTPELNEILDTFLNDECLLHAM
HISTGLSIFDTSLF
(SEQ ID NO: 7)
gatgcactcgatgattttgacctcgatatgcttgggagtgatgcgctcgatgacttcgatttggatatgcttggatctgatgcc
ctcgacgatttcgaccttgatatgctcgggtcagacgctttggatgactttgaccttgacatgctggggagcggctcccggga
gatggctgacacagtaataccccaaaaagaggaggctgcgatttgtgggcagatggatttgtcccaccctccaccgagagg
tcatcttgacgaattgacaacgacgctcgaatccatgaccgaggacctgaacctcgatagcccgctcacccccgagttgaat
gagatcctggatacatttcttaatgatgagtgtttgcttcacgcaatgcatatttctacgggtcttagtattttcgacacgagcc
tgttt

Plasmid Cloning

The new activation moieties (AMs) were synthesized by IDT and cloned into NUC9-dSaCas9 vector. The fusion proteins were expressed from the EFS promoter.

sgRNA sequence used:
MYD88-1;
(SEQ ID NO: 35)
GGTTCATACGGTCCTGCCCTC
MYD88-2;
(SEQ ID NO: 36)
GGAGCCACAGTTCTTCCACGG
MYD88-3;
(SEQ ID NO: 37)
CTCTACCCTTGAGGTCTCGAG
FGF21-1;
(SEQ ID NO: 38)
TGCCAGATTCCAGTTGTCCAG
FGF21-2;
(SEQ ID NO: 39)
ACATTCCTGAGTCTCAGAGAG
FGF21-3;
(SEQ ID NO: 40)
GGCTAATTTCCTGGAGCCCCT
GCG-1;
(SEQ ID NO: 41)
CTGTGAGGCTAAACAGAGCTG
GCG-2;
(SEQ ID NO: 42)
GTCTCTCACCCAATATAAGCA
GCG-3;
(SEQ ID NO: 43)
AAATCACTTAAGTTCTCTAAA

Cell Transfection

HEK293FT cells were plated on 24-well plate at 75,000 cells per well. 250 ng of fusion protein expressing plasmids NUC9-dsaCas9-AM were co-transfected with sgRNA expressing plasmids LvSG03 using Lipofectamine 2000 according to manufacturer's instruction. After 24 hours, transfected cells underwent puromycin selection, and harvested the next day.

dSaCas9 nucleotide sequence;
(SEQ ID NO: 28)
atgaagcggaactacatcctgggcctggccatcggcatcaccagcgtggg
ctacggcatcatcgactacgagacacgggacgtgatcgatgccggcgtgc
ggctgttcaaagaggccaacgtggaaaacaacgagggcaggcggagcaag
agaggcgccagaaggctgaagcggcggaggcggcatagaatccagagagt
gaagaagctgctgttcgactacaacctgctgaccgaccacagcgagctga
gcggcatcaacccctacgaggccagagtgaagggcctgagccagaagctg
agcgaggaagagttctctgccgccctgctgcacctggccaagagaagagg
cgtgcacaacgtgaacgaggtggaagaggacaccggcaacgagctgtcca
ccaaagagcagatcagccggaacagcaaggccctggaagagaaatacgtg
gccgaactgcagctggaacggctgaagaaagacggcgaagtgcggggcag
catcaacagattcaagaccagcgactacgtgaaagaagccaaacagctgc
tgaaggtgcagaaggcctaccaccagctggaccagagcttcatcgacacc
tacatcgacctgctggaaacccggcggacctactatgagggacctggcga
gggcagccccttcggctggaaggacatcaaagaatggtacgagatgctga
tgggccactgcacctacttccccgaggaactgcggagcgtgaagtacgcc
tacaacgccgacctgtacaacgccctgaacgacctgaacaatctcgtgat
caccagggacgagaacgagaagctggaatattacgagaagttccagatca
tcgagaacgtgttcaagcagaagaagaagcccaccctgaagcagatcgcc
aaagaaatcctcgtgaacgaagaggatattaagggctacagagtgaccag
caccggcaagcccgagttcaccaacctgaaggtgtaccacgacatcaagg
acattaccgcccggaaagagattattgagaacgccgagctgctggatcag
attgccaagatcctgaccatctaccagagcagcgaggacatccaggaaga
actgaccaatctgaactccgagctgacccaggaagagatcgagcagatct
ctaatctgaagggctataccggcacccacaacctgagcctgaaggccatc
aacctgatcctggacgagctgtggcacaccaacgacaaccagatcgctat
cttcaaccggctgaagctggtgcccaagaaggtggacctgtcccagcaga
aagagatccccaccaccctggtggacgacttcatcctgagccccgtcgtg
aagagaagcttcatccagagcatcaaagtgatcaacgccatcatcaagaa
gtacggcctgcccaacgacatcattatcgagctggcccgcgagaagaact
ccaaggacgcccagaaaatgatcaacgagatgcagaagcggaaccggcag
accaacgagcggatcgaggaaatcatccggaccaccggcaaagagaacgc
caagtacctgatcgagaagatcaagctgcacgacatgcaggaaggcaagt
gcctgtacagcctggaagccatccctctggaagatctgctgaacaacccc
ttcaactatgaggtggaccacatcatccccagaagcgtgtccttcgacaa
cagcttcaacaacaaggtgctcgtgaagcaggaagaagccagcaagaagg
gcaaccggaccccattccagtacctgagcagcagcgacagcaagatcagc
tacgaaaccttcaagaagcacatcctgaatctggccaagggcaagggcag
aatcagcaagaccaagaaagagtatctgctggaagaacgggacatcaaca
ggttctccgtgcagaaagacttcatcaaccggaacctggtggataccaga
tacgccaccagaggcctgatgaacctgctgcggagctacttcagagtgaa
caacctggacgtgaaagtgaagtccatcaatggcggcttcaccagctttc
tgcggcggaagtggaagtttaagaaagagcggaacaaggggtacaagcac
cacgccgaggacgccctgatcattgccaacgccgatttcatcttcaaaga
gtggaagaaactggacaaggccaaaaaagtgatggaaaaccagatgttcg
aggaaaagcaggccgagagcatgcccgagatcgaaaccgagcaggagtac
aaagagatcttcatcaccccccaccagatcaagcacattaaggacttcaa
ggactacaagtacagccaccgggtggacaagaagcctaatagagagctga
ttaacgacaccctgtactccacccggaaggacgacaagggcaacaccctg
atcgtgaacaatctgaacggcctgtacgacaaggacaatgacaagctgaa
aaagctgatcaacaagagccccgaaaagctgctgatgtaccaccacgacc
cccagacctaccagaaactgaagctgattatggaacagtacggcgacgag
aagaatcccctgtacaagtactacgaggaaaccgggaactacctgaccaa
gtactccaaaaaggacaacggccccgtgatcaagaagattaagtattacg
gcaacaaactgaacgcccatctggacatcaccgacgactaccccaacagc
agaaacaaggtcgtgaagctgtccctgaagccctacagattcgacgtgta
cctggacaatggcgtgtacaagttcgtgaccgtgaagaatctggatgtga
tcaaaaaagaaaactactacgaagtgaatagcaagtgctatgaggaagct
aagaagctgaagaagatcagcaaccaggccgagtttatcgcctccttcta
caacaacgatctgatcaagatcaacggcgagctgtatagagtgatcggcg
tgaacaacgacctgctgaaccggatcgaagtgaacatgatcgacatcacc
taccgcgagtacctggaaaacatgaacgacaagaggccccccaggatcat
taagacaatcgcctccaagacccagagcattaagaagtacagcacagaca
ttctgggcaacctgtatgaagtgaaatctaagaagcaccctcagatcatc
aaaaagggctaa
tracrRNA sequence;
(SEQ ID NO: 30)
guuuuaguacucuggaaacagaaucuacuaaaacaaggcaaaaugccgug
uuuaucacgucaacuuguuggcgagauuuuuuu

RNA Isolation and Gene Expression Analysis

For gene expression analysis, the transfected cells were harvested at 48-72 h after transfection and lysed in RLT buffer to extract total RNA using RNeasy kit (Qiagen).

For Taqman analysis, 1 μg of total RNA was used to generate cDNA using TaqMan ™High-Capacity RNA-to-cDNA Kit (Applied Biosystems) in 10 μl volume. The generated cDNA was diluted 10 fold and 3.33 μl was used per Taqman reaction (10 μL total volume per reaction). Taqman reaction was run using Taqman gene expression master mix (ThermoFisher) in Roche LightCycler 96 or LightCycler 480 and analyzed using LightCycler 96 analysis software.

Taqman probe product IDs:

MYD88; Hs01573837_g1 (FAM)

FGF21: Hs00173927_ml

GCG: Hs01031536_ml

HPRT: Hs99999909_ml (VIC PL)

Taqman QPCR condition:

Step 1; 95° C. for 10 min

Step 2; 95° C. for 15 sec

Step 3; 60° C. for 30 sec

Repeat Step 2 and 3; 40 times

Result

FIG. 1. The structure of AAV vector and the ten activation moieties

Our AAV vector contains dSaCas9 fused with activation moieties shown in the below diagram. The fusion proteins are expressed by the EFS promoter, and sgRNA is expressed from the U6 promoter. Seven new activation moieties were created; VP64-MyoD, VP64-HSF1, VP32-p65, VP64-miniRTA, VP64-microRTA, VP64-p65 and VPH. The reported activation moieties (VP64, VP160 and VPR) were also tested for comparison. The size limit of AAV vector is 5 kb, and the components add up to 4.45 kb, which leaves room for the fused activation moieties around 550 bps. Thus the following seven activation moieties fit within the vector size limit; VP64, Vp160, VP64-MyoD, VP64-HSF1, VP32-p65, VP64-miniRTA and VP64-microRTA.

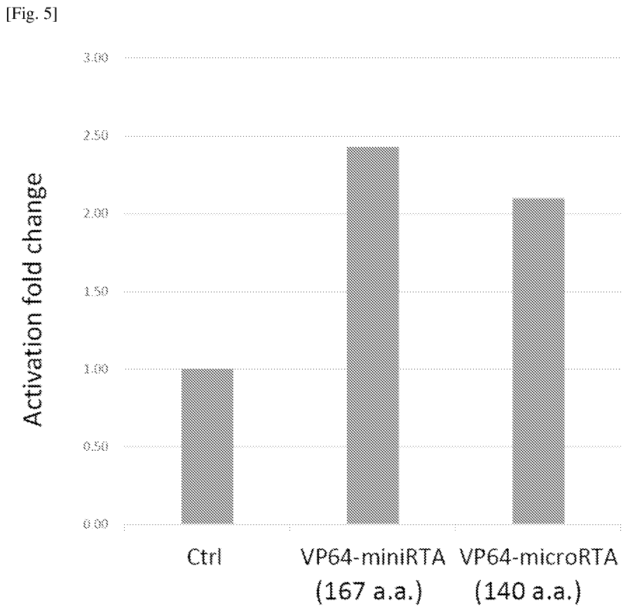

FIG. 2. MYD88 gene activation by the nine activation moieties

The activation function of the six new activation moieties were tested with three different sgRNAs (MYD88-1, -2 and -3) targeting the human MYD88 promoter region. The three activation moieties, VP64, VP160 and VPR were also tested for comparison. In all the three sgRNAs tested, VP64-RTA showed the best gene activation of the six moieties fit within the AAV vector size limit.

FIG. 3. FGF21 gene activation by the nine activation moieties

The activation function of the six new activation moieties were tested with three different sgRNAs (FGF-1, -2 and -3) targeting the human FGF21 promoter region. The three activation moieties, VP64, VP160 and VPR were also tested for comparison. In all the three sgRNAs tested, VP64-RTA showed the best gene activation of the six moieties fit within the AAV vector size limit.

FIG. 4. GCG gene activation by the nine activation moieties

The activation function of the six new activation moieties were tested with three different sgRNAs (GCG-1, -2 and -3) targeting the human GCG promoter region. The three activation moieties, VP64, VP160 and VPR were also tested for comparison. In all the three sgRNAs tested, VP64-RTA showed the best gene activation of the six moieties fit within the AAV vector size limit.

FIG. 5. MyD88 gene activation by VP64-miniRTA and VP64-microRTA

The activation function of VP64-miniRTA (164 a.a.) and VP64-microRTA (140 a.a.) were compared in human MYD88 promoter. VP64-microRTA showed similar level of activation as VP64-miniRTA. gMYD88_2 was used.

### CONCLUSION

Our VP64-miniRTA (miniVR; 167 a.a., 501 bps) and VP64-microRTA (microVR; 140 a.a., 420 bps) are small enough to fit within the size limit of AAV vector (5 kb) in the presence of other elements such as Cas9, sgRNA and promoters.

Thus, VP64-miniRTA and VP64-microRTA are powerful moieties to use with CRISPR technology and AAV delivery system.

This application is based on U.S. provisional patent application Ser. No. 62/715,432 (filing date: Aug. 7, 2018), the contents of which are incorporated in full herein by this reference.

1. A transcription activator consisting of not more than 200 amino acids and comprising VP64 and a transcription activation site of RTA.

2. The transcription activator according to claim 1, wherein said VP64 comprises
(1) the amino acid sequence shown in SEQ ID NO: 1,
(2) the amino acid sequence of (1) wherein 1 or several amino acids are deleted, substituted and/or added, or
(3) an amino acid sequence 90% or more identical to the amino acid sequence of (1).

3. The transcription activator according to claim 1, wherein said transcription activation site of RTA comprises
(4) the sequence shown in SEQ ID NO: 2,
(5) the sequence shown in SEQ ID NO: 3,
(6) the amino acid sequence of (4) or (5) wherein 1 or several amino acids are deleted, substituted and/or added, or
(7) an amino acid sequence 90% or more identical to the amino acid sequence of (4) or (5).

4. A complex comprising a nucleic acid sequence-recognizing module specifically binding to a target nucleotide sequence in a double-stranded DNA and the transcription activator of claim 1 bonded to each other, and activating transcription of a targeted gene in the DNA.

5. The complex according to claim 4, wherein said nucleic acid sequence-recognizing module comprises a CRISPR effector protein lacking the ability to cleave at least one strand of the double-stranded DNA.

6. The complex according to claim 5, wherein said CRISPR effector protein lacks the ability to cleave both strands of the double-stranded DNA.

7. The complex according to claim 5, wherein the CRISPR effector protein is derived from Staphylococcus aureus or Campylobacter jejuni.

8. A nucleic acid encoding the transcription activator according to claim 1.

9. A nucleic acid encoding the complex according to claim 4.

10. A vector comprising the nucleic acid according to claim 8.

11. The vector according to claim 10, wherein said vector is an adeno-associated virus vector.

12. A method for activating transcription of a targeted gene in a cell, comprising a step of introducing the complex according to claim 4 into the cell.

13. The method according to claim 12, wherein the cell is a mammalian cell.

14. The method according to claim 13, wherein said mammal is a human.
